(19) United States
(12) Patent Application Publication
(10) Pub. No.: US 2024/0057925 A1
(43) Pub. Date: Feb. 22, 2024

(54) Health Monitoring and Management System Using Ectopic Beats as Early Cardiac Health Marker

(71) Applicant: LifeQ B.V., Amsterdam (NL)

(72) Inventors: Franco Du Preez, Cumming, GA (US); Wikus Theo Villet, Kuilsrivier (ZA); Janina de Wit, Bellville (ZA); Melissa Kistner, Stellenbosch (ZA); Tom deLaubenfels, Atlanta, GA (US); Etienne Terblanche, Alpharetta, GA (US); Laurence Richard Olivier, Alpharetta, GA (US)

(21) Appl. No.: 18/259,498

(22) PCT Filed: Dec. 30, 2021
(86) PCT No.: PCT/US2021/065709

§ 371 Date: Jun. 27, 2023

Related U.S. Application Data
(60) Provisional application No. 63/132,124, filed on Dec. 30, 2020.

(51) Int. Cl. A61B 5/364; A61B 5/00; A61B 5/366; A61B 5/363; A61B 5/024

(52) U.S. Cl. CPC A61B 5/364; A61B 5/7275; A61B 5/7264; A61B 5/721; A61B 5/7221; A61B 5/7225; A61B 5/7239; A61B 5/7282; A61B 5/002; A61B 5/0022; A61B 5/4812; A61B 5/4809; A61B 5/4842; A61B 5/4848; A61B 5/366; A61B 5/363; A61B 5/02444; A61B 5/725; A61B 5/746; A61B 2562/0219

(57) ABSTRACT

A method for health monitoring is provided including at least one non-invasive wearable device capable of collecting and storing data, and external monitoring devices that displays and analyses the data for accurate monitoring and anomaly detection. The wearable devices are configured to collect low-latency PPG-derived bio signals and can utilize multiple devices for accuracy and continuity. The system may further include a dashboard that analyzes the information as well as displaying basic information such as number of beds (in-use, available), staff-to-patient ratio, etc. The data can be collected and accessed remotely and may be utilized before, during, and/or after a patient is dispatched from a clinical setting.

### CROSS-REFERENCE TO RELATED APPLICATIONS

This application claims the benefit of U.S. Provisional Patent Application No. 63/132,124, filed Dec. 30, 2020, which is incorporated by reference herein in its entirety.

### BACKGROUND

Ectopic beats are well described and occur when the heart contracts due to a signal that originates outside of the heart's native pacemaker. This causes a premature contraction cycle and can broadly originate either on the upper chambers (atria) as normal beats do, or alternatively from the large lower chambers of the heart (ventricles). When several such abnormal pulses follow one another, the heart is said to be in a state of arrhythmia.

Ectopic beats are normal when occurring at very low frequencies in healthy individuals, but there is a great amount of variation in their frequency that would be classified as healthy between different individuals. A healthy individual may move closer to a state of heart disease due to any combination of factors. Factors that change the ectopic beat frequency can be described from several perspectives, with probably the most obvious perspectives being: (1) the type of causative agent that is bringing the change about (i.e., lifestyle and environmental factors, genetic factors, or infectious agents), and (2) the timescale over which these changes take place, such as decades (e.g., ageing, smoking), years or months (e.g., chronic disease such as hypertension), days (e.g., acute infectious disease such as Covid-19), hours (e.g., behavioral, such as dehydration, alcohol), or hours to minutes (e.g., emergencies, such as clinical decompensation).

A clear example that illustrate such a breakdown, is the natural ageing process, which slowly increases the number of ectopic beats due to structural changes or damage to the heart muscle (i.e., timescale of decades, lifestyle and genetic causes), to faster changes due to noncommunicable chronic diseases such as uncontrolled high blood pressure (i.e., timescale of years, lifestyle and genetic causes), to even faster changes due to communicable diseases such as a Covid-19 infection that can lead to a temporarily increased frequency of ectopic beats and arrhythmias (i.e., timescale of days, reversible, infectious agent).

Chronic conditions that lead to arrhythmias, such as atrial fibrillation (AFib), which consist of runs of many ectopic beats in succession (i.e., Atrial Premature Beats), are especially prone to causing changes in the frequency of ectopic beats.

In the extreme case, where ectopic beat frequencies are high, clinical thresholds for the frequency of different types of ectopic beats exist and have been examined separately for atrial and ventricular premature beats. Above these threshold values, further medical investigation is performed to understand the optimal route of treatment, which includes a wide range of treatment options for patients suffering arrhythmias, including pacemaker device implants and drug-based therapies with agents that alter the heart's conduction of electric signals, such as ion channel blockers.

Presently, no technology is widely implemented for doing longitudinal monitoring of ectopic beats to quantify their frequency or to track the evolution of their frequency or other characteristics over time in human participants in a way that is unobtrusive and tolerated over several years by monitored subjects. This is mainly due to the lack of data regarding a person's physiological state while they are not being monitored by a human or an invasive, oftentimes expensive device.

Current, continuous monitoring systems are not cost-effective, scalable, or an accurate way to monitor a person continuously and in near real-time. Apart from lifestyle fitness trackers and mobile applications, there is very little physiological data collected from healthy people prior to hospitalization or an alert of a health concern. This type of information can be instrumental in prevention and recovery in health and wellbeing. For example, when someone has a heart attack or a stroke, there are usually prior physiological indicators that were not monitored or observed, such as ectopic beats, that would have been useful for a doctor to know of. Another problem with the current state of the art is that most elderly care systems utilize an SOS button to alert caregivers and healthcare providers that there is an emergency. These can cause delay, false positives, and a need for the user to be conscious/aware of their situation. Further, it is not particularly helpful to collect data from a healthy individual that is not in a format that can be used for a baseline analysis and then collect information when the individual is sick without a healthy baseline to which it can be compared.

When someone goes to a primary care physician (PCP) for their yearly checkup, the PCP is only equipped with observable data during the time that the patient is in the doctor's office. This type of patient monitoring is rare and sporadic, like a series of snapshots rather than continuous monitoring. There is no method for constantly monitoring and collecting information about an individual, let alone, many people simultaneously and continuously. As the use of telehealth increases, so too must the information available to doctor's remotely improve in breadth and accuracy.

### BRIEF SUMMARY

The present invention provides a means through an Internet of Things (IOT) wearable to detect slow changes and an initial baseline frequency of ectopic beats. Both the starting baseline frequency and trends of an increasing ectopic beat frequency can be used to quantify the very early stages heart disease and the present invention can be used to notify clinical professionals to intervene early in disease development through a variety of means that can slow the progression of subclinical heart disease. This can be understood as an instance of a preventative health solution and this instance aims to continuously monitor a person's ectopic beats across-time.

The present invention longitudinally monitors (i.e., monitoring continuously over a long period of time) ectopic beats to quantify their frequency or to track the evolution of their frequency or other characteristics over time in human participants in a way that is unobtrusive and tolerated over several years by monitored subjects. The present invention will use ectopic beats in this manner as a biomarker for changes in the heart muscle and changes to health status, as well as the development of specific diseases, such as arrhythmia conditions.

The invention relies on a non-invasive device for following the peripheral pulse of an individual and uses this information to determine the timing of individual heartbeats as well as the timing between successive beats. This information is recorded when sufficient signal quality is available, which might not be possible under conditions of vigorous motion, but which is feasible for most of the time under sedentary and resting conditions, including sleep. Anomalous beat timings that do not follow a regular pattern are marked as Ectopic Beats. Depending on the specific case, it could be possible to deduce whether an Ectopic Beat is likely to have originated from either the atria or ventricles of the heart. It is also possible to measure via pulse signal amplitude and intensity, cases where mechanical beats of the heart are weakened or absent and lead to reduced perfusion and temporarily lowered blood pressure. In such cases, the weakened peripheral pulse lead is known as a pulse deficit and it is more often seen with ectopic beats that have a ventricular origin (Premature Ventricular Contractions or PVCs) than an atrial origin (Premature Atrial Contractions or PACs), a temporary interruption in cardiac output by the heart can increase the risks of suffering a stroke due to blood pooling and clotting, but this can also lead to syncope. Many modern wearables have the capability to measure ECG when a user touches her finger to a specific part of the wearable device on the opposite arm. The invention therefore has the capability for alerting an end user under different circumstances based on readings from the peripheral pulse and one example would be to request an ECG recording when a condition such as tachycardia is recorded to determine whether the tachycardia is a broad complex ECG 319 (more dangerous, requiring immediate medical attention) or narrow complex ECG 321.

Ectopic beats are also of interest for wellness and early disease prevention as they are very early markers of disease processes that could lead to the development of arrhythmias such as Atrial Fibrillation. Early feedback to patients or end-users regarding the burden of ectopic beats as well as any changes to the number of these beats as revealed by trend analysis, or changes to the number of sites in the heart that generate such beats, could be useful in alerting wearers to the need for pursuing a lifestyle that will promote increased heart health, such as avoiding saturated fats and cholesterol in the diet and participating in regular aerobic exercise. It might also help healthcare providers to gain awareness of early stages of arrhythmia development and motivate them to find other comorbidities that might be the reason for a decline in cardiac health, such as obstructive sleep apnea. Such a comorbidity could be treated with a simple CPAP, BiPAP or AutoPAP device that helps to open the airway of sufferers. Similarly, predisposing conditions such as high blood pressure could also put unnecessary stress on the cardiovascular system to promote changes leading to arrhythmia and is very treatable.

By recording the context of a person's beats not only the peripheral pulse, but also demographic factors such as height, weight, age and gender of a subject via user interaction, as well as sleep and activity via wearable sensor data streams (peripheral pulse via e.g. PPG and motion via actigraphy using an accelerometer) and medical context via integration with electronic health records, it becomes possible to implement algorithms that discover relationships between lifestyle factors and changes to the type and number of ectopic beats seen in a particular individual. This opens up the possibility of discovering personalized and unique causes of increases and or decreases in the frequency of ectopic beats in a monitored individual and could be used to determine whether certain medications might predispose a wearer to arrhythmias or whether certain lifestyle interventions contribute more to heart health for the particular monitor individual than others. Such a system can also provide automated recommendations to improve heart health and where outcomes are measured while data-driven behavioral recommendations are applied, the system can be described as a closed loop health monitoring system.

Technology for analyzing such time series data of the peripheral pulse and discovering associations with lifestyle, medical and demographic factors is quickly evolving and many supervised and semi-supervised machine learning algorithms are available for training on such data streams to determine where and which type of associations are prevalent. Having access to a cohort of individuals with different levels of ectopic burden, who are wearing both ECG recording devices and peripheral pulse recording devices provides the raw material that such machine learning algorithms can be trained on.

A key information stream that reveals the nature of a specific peripheral beat as being ectopic or normal (as in originating from a pulse created by the heart's pacemaker or sinoatrial node) is the timing of individual heartbeats. The pacemaker produces heartbeats with a well-known and characteristic signature which can be analyzed with several tools available for calculating heart rate variability (HRV), such as the time domain (e.g., SDNN), frequency domain (e.g. HF/LF ratio) and entropy based calculations (e.g. sample entropy). Typically, a high frequency component is present which is caused by the breathing process, since pressure on the heart changes during the breathing cycle, which leads to differences in filling volume of the atria. This phenomenon is known as sinus arrhythmia. Ectopic beats originate from abnormal electrical activity in heart tissue that is often diseased, and this tissue could be in either the atria or ventricles (causing PACs or PVCs respectively). It is also possible for the origin of these abnormal ectopic beats to be localized in the heart at a single or multiple sites. When this tissue produces an action potential and conduction of an electrical impulse across the myocardium, it is possible for the impulse to enter and reset the pacemaker cells of the heart, which changes the phase of individual heart beats (often seen with PACs since the pacemaker cells are in the right atrium) or alternatively, it is also possible for such an impulse to not reset the pacemaker cells if it arrives at a time when the cells are refractory to electrical impulses (often the case with PVCs). It is possible to train machine learning algorithms, or to create statistical models that can determine whether the timing of a series of heartbeats are indicative of a reset of the pacemaker cells (beat to beat timing variation in line with that expected from the sinoatrial node) or whether it appears that the pacemaker cells have not been reset, while changes to the peripheral pulse occurred (e.g. it's amplitude disappearing during a PVC where the heart pumps less effectively due to the slow and unnatural electrical conduction pattern from ventricles to atria).

In some cases, a run of multiple ectopic beats can regularly occur. This is the case with conditions such as bigeminy and trigeminy. In this case ectopic beats can trigger follow up ectopic beats from other sites in the heart with a predetermined pattern. When plotting successive heartbeats using a Poincare plot, such runs are revealed as concentrated points on a plot of beat timing (x-axis) vs successive beat timing (y-axis). When changes in the tissue that generate ectopic beats occur, the pattern seen in the Poincare can change over time to reveal the presence of new or fewer sites in the heart that generate ectopic beats. Capturing these changes visually can be substituted with a supervised or unsupervised machine learning approach using the same data as described above.

The system of the present invention is configured to perform non-invasive health monitoring by acquiring data on the peripheral pulse of a subject through the use of non-invasive wearable, analyzing the data collected by extracting and classifying heart beats using a predictive algorithm, predicting other contextual information using data from the wearable device, and analyzing trends in ectopic heart beat frequency to share the ectopic beat frequency and trends from the analysis with patients and care providers. In an aspect, the wearable device includes at least one microcontroller, and sensors capable of monitoring the peripheral pulse. The sensors can include photoplethysmography (PPG), seismocardiography (SCG), ballistocardiography (BCG), impedance cardiography (ICG), and/or electrodermal activity (EDA) sensors. In an aspect, these sensors can be replaced by ECG to follow beat timing via the electric signals of the heart. The wearable device can also include contextual sensors, such as, but not limited to, triaxial accelerometer, gyroscopic sensors, and/or electromyography sensors.

In addition, the system, via the wearable device, or another electronic device, can obtain optional demographic information of a subject, including, but not limited to, height, weight, BMI, sex, and age. In addition, optional medical history of a subject by means of Electronic Health Record of similar can be included. The wearable device can also include a communication module for sending recorded data to a computing device connected to the internet. The communication module can be configured to transmit the acquired data to one or a series of computing device, such as, but not limited to, mobile phones, servers, tablet computers, the computing device connecting to either the internet, or a local communication network, such as bluetooth or wifi, that can reach the internet through other devices.

The analysis performed by the system of the acquired data can be done on the wearable device and/or a computing device connected to the internet. The systems and method can use the predictive algorithm to identify the extracted heartbeats as normal beats, initiated by the sinoatrial node or pacemaker of the heart, ectopic beats, not initiated by the sinoatrial node or pacemaker of the heart, or undetermined beats. The system and method can also utilize other contextual information, as well as optimal demographic data, including, but not limited to, sleep state and sleep stages, activity patterns, and/or fitness level to assist in the classification of the heartbeats. In an aspect, the system can also store and analyze trends in ectopic heart beat frequency to determine the ectopic heart beat frequency and trends for the wearer of the wearable device. These trends can then be shared with the patients to inform them on their progress toward improved or worsening heart health at the earliest or later stages of disease. They can also be shared to patients, to warn them of runs of ectopic beats associated with a reduced pulse or pulse deficit to be used for, but not limited to, timely visit to the ER and/or reach out to their physician. For runs of ectopic beats detected in athletes during sleep are used to enable early detection of atrial fibrillation that only manifests under conditions of high vagal tone, such as deep sleep.

This information can also be shared with care providers to track disease progress, treatment efficiency, and lifestyle intervention efficiency, as well as to alert with regard to periods of pulse deficit that predispose the subject to stroke or syncope, and to show historical periods of pulse deficit to determine whether a stroke might have been caused by runs of ectopic beats/arrhythmia.

In an aspect, the total number of ectopic beats or trends in the historic frequency of ectopic beats in a subject are can be shared with clinicians to inform patient screening by highlighting patients that have a high or increasing ectopic burden and monitor the condition of the subject. The monitoring can include, but is not limited to, monitoring the efficiency of treatment, monitoring the number of arrhythmogenic sites, and/or monitoring the compliance and efficiency of lifestyle interventions such as exercise through the data recorded by the wearable device, or other related device. In an aspect, the total number of ectopic beats or trends in the historic frequency of ectopic beats in a subject are can be shared with a subject or patient, to inform them of information including ectopic burden with threshold alerts to seek medical care, trends in ectopic burden with threshold alerts to seek medical care, and/or the relationship between the metadata, ectopic burden, and ectopic trends to improve wellness. The metadata can include, but is not limited to, sleep parameters, exercise and activity parameters such as, but not limited to number and amount of exercise per week, sedentary behavior such as longest consecutive sitting, time per day, and steps taken per day, as well as body weight.

In an aspect, the devices capable of doing the analysis discussed above can be configured to determine whether tachycardia is present from the peripheral pulse rate using digital signal processing techniques, alert the user to record an ECG using the wearable device by touching a finger to an electrode on the device when tachycardia is detected, and determine from the ECG data whether the QRS complex is a broad or narrow complex. In addition, the devices can be configured to alert emergency services, the patient, and care provider of the presence of ventricular tachycardia in the case of broad complex QRS.

In an aspect, the system and methods can utilize an algorithm that takes as input the number of ectopic beats, trends in the number of ectopic beats, and features describing individual ectopic beats, such as, but not limited to the pulse attenuation or pulse deficit. From this, the algorithm can produce a prediction for the cause of ectopic beats in cases where these are elevated, or changing or trending over time, and/or where the potential causes include, but are not limited to, heart enlargement or other myocardial abnormalities, changes in potassium level, and decreased blood supply to the heart (ischemic disease).

In an aspect, the wearable device/computing device can also be configured to use signal acquisition parameters, including, but not limited to, amplifier gain and LED current which are adjusted dynamically during measurement through a closed loop controller to continuously optimize the signal to noise ratio for the peripheral pulse signal. In addition, the wearable device/computing device can be configured to convert the peripheral pulse signal to a unit that would retain continuity in the peripheral pulse signal across signal acquisition parameter adjustments. These units can include, but are not limited to, (i) PPG, where a unit would be the ratio between light emitted by the photodiode and light received; (ii) BCG, where a unit would be acceleration measured in G; (iii) impedance, where a unit would be the complex resistance for a given frequency; and/or (iv) galvanic skin response, where a unit would be the resistance.

In an aspect, the system and methods can be configured to filter the signal to remove low quality recordings. This can be done by using readings from contextual sensors to determine whether motion is present, which would distort signals, using readings from peripheral pulse sensors to determine whether the signal to noise ratio is acceptable, and/or using a threshold for detecting discontinuities in the peripheral pulse signal that could be due to adjustments in the signal amplification parameters or due to noise sources. The signal to noise ratio can be determined through any of the following means of comparing the ratio of high frequency sample by sample noise to signal in frequency bands corresponding to measured heart rate, or ratio of signal energy in the frequency bands corresponding to heart rate to other frequency bands. The signal can be filter to remove high frequency noise by application of a low pass filter that removes sample by sample noise and low frequency noise by application of a low pass filter that removes noise that includes, but is not limited to physiological processes, such as breathing. Band-pass filtering the signal can be used to remove the low and high frequency noise in one step.

In an aspect, the system is further configured to segment individual peripheral pulses from the peripheral pulse signal readings by taking a derivative of the peripheral pulse and using zero crossings to locate peaks and/or troughs of the signal, and taking a second order derivative of the peripheral pulse to locate inflection points. In an aspect, the time resolution of pulse peaks and/or troughs is increased beyond the time resolution of the sampling rate by performing interpolation of the signal and finding the peak or trough in the interpolated signal through a polynomial interpolation and a spline interpolation.

In an aspect, the system and methods can reduce individual pulses to a set of features relevant for discriminating ectopic beats from beats originating from the sinoatrial node, the set of features including beat-to-beat timing, beat amplitude in the units, absolute signal intensity in units for the trough of the signal, heart rate variability based on this and surrounding beats for approximately one minute, and pulse waveform features.

In an aspect, the algorithm utilized by the system and method for classifying individual beats as ectopic or normal is based on any of the following principles: a supervised machine learning model trained against ectopic beats identified in datasets scored by expert human or algorithm, where said datasets include simultaneous ECG and PPG recordings, a semi-supervised machine learning model, which produces clusters in the space of the Poincare plot (beat compared to previous beat) and which marks the cluster(s) closest to the diagonal as normal sinoatrial beats, while marking other clusters as ectopic beats; or a Probabilistic Graphical Model (PGM) that models the distribution of beat timing for normal sinoatrial beats as well as for ectopic beats. The supervised algorithm is trained on population level data and/or data collected from an individual to provide personalized training of the model.

In an aspect, a variation of the PGM can have the distribution for normal and ectopic beats depends on one or more earlier beats, the PGM is a Hidden Markov Model (HMM) with at least one hidden state representing normal and another hidden state representing ectopic beats, or the PGM is a Bayesian network, where the distribution for normal and ectopic beats depends on at least the previous beat. In any of the cases above, the maximum likelihood is used to predict the nature (normal or ectopic) of the next beat to beat timing. In addition, to its status as normal or ectopic, the following information is predicted from the features available for each beat: whether the beat is likely to have originated from either the atrium or ventricle of the heart; and the ectopic beat path, in the case of the beat classification algorithm being unsupervised, from which the beat originates, with new clusters potentially representing new arrhythmogenic modes in the heart muscle.

### BRIEF DESCRIPTION OF THE DRAWINGS

FIG. 1 is a flowchart illustrating the steps of detecting ectopic beat types and their influence on the peripheral pulse waveform according to an aspect of the present invention.

FIG. 2 is a flowchart illustrating the digital signal processing and extraction of pulse waveform features according to an aspect of the present invention.

FIG. 3 is a block diagram illustrating algorithms for classifying beat-to-beat intervals according to an aspect of the present invention.

FIG. 4 is a schematic representation of the monitoring and management system according to an aspect of the present invention according to an aspect of the present invention.

FIG. 5 is an overview of the monitoring and management system using a non-invasive peripheral pulse monitoring device according to an aspect of the present invention.

### DETAILED DESCRIPTION

The following description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples and are not limited to those set forth herein and may be changed as will be apparent to one of ordinary skill in the art. Description of functions and constructions that are well known to one of ordinary skill in the art may be omitted for increased clarity and conciseness.

The present invention is aimed at longitudinal monitoring a human subject to record the pattern in their Ectopic Beats. This is done using a non-invasive wearable device that can detect ECG signals. The ectopic beats are then analyzed for source of origin, intensity, etc. This data may be vital for early detection of heart diseases, prevention, and determination of the appropriate treatment/effectiveness of such treatment.

The wearable device collects the subject's data including contextual data such as, but not limited to, the activity of the user and biological information. The ectopic beat types are then analyzed using several measures of heart variability. The wearable device can also include various sensors utilized in collecting physiological data of the user. In an aspect, the wearable device can include pulse sensors used to collect pulse sensor readings. These pulse sensor readings can be monitored, and the sensor circuit parameters are updated to provide a more continuous and interpretable sensor reading. Once the data is collected, there are various algorithms, discussed below, that may be used to classify the beat-to-beat intervals to distinguish between normal and ectopic beats. Using this information, several methods for leveraging the data in early monitoring and management of cardiac health exist. These routes may include screening for specific underlying causes of ectopic beat frequencies and trends, screening for and managing runs of ectopic beats, determining behavioral and environmental factors that influence ectopic beat frequencies, and opportunistically prompting the collection of ECG information on the wearable device 317.

### Non-Invasive Peripheral Pulse Monitoring Device

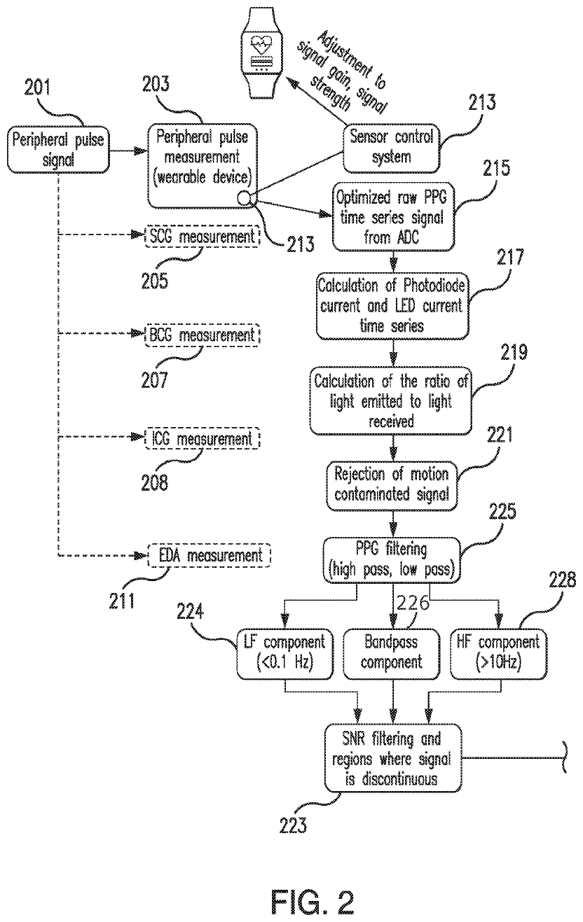

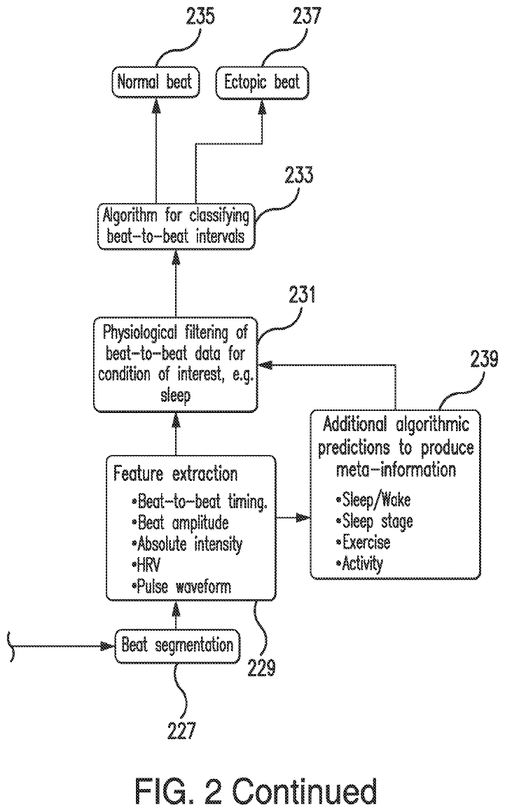

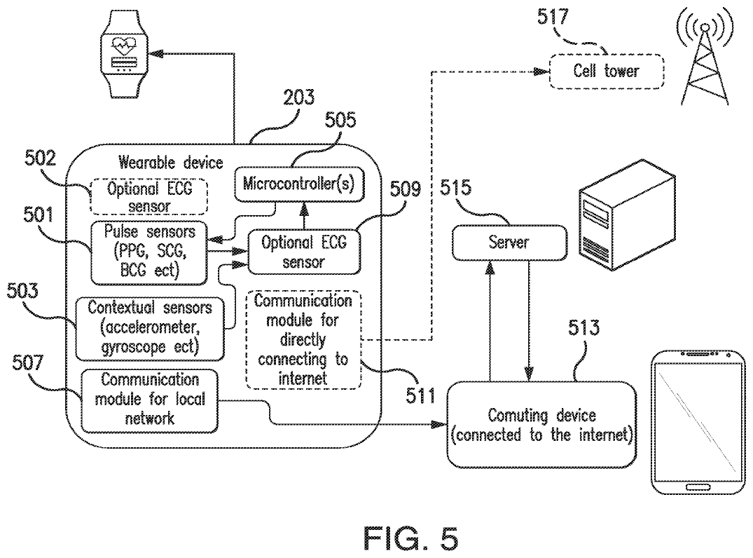

As discussed above, the system relies on a wearable device with sensing means. In an aspect, the wearable device includes a non-invasive peripheral pulse monitoring device 203 (see FIGS. 2 and 5), which tracks the peripheral pulse signal 201 of the user wearing the device 203 via various pulse sensors 501. In an aspect, the wearable device 203 can also include an ECG sensor 502. Several technologies are available for tracking the pulses of blood in the peripheral blood vessels, with the most used technology at time of writing being pulse plethysmography (PPG) 501. Several other technologies can also be used to record time series data that fluctuate in rhythm with the peripheral pulse including, but not limited to, seismocardiography (SCG), ballistocardiography (BCG), impedance cardiography (ICG), and electrodermal activity (EDA) and each of these are viable alternatives to PPG. It is preferable that the technologies be able to record time series data that fluctuate in rhythm with the pulse of heart, and more preferably with the peripheral pulse, of the user.

To effectively monitor the peripheral pulse, a sensor control system 213/509 can be loaded as a program on the processors/microcontroller(s) 505 of the wearable device 203 for controlling the signal acquisition parameters, as shown in FIGS. 2 and 5. Such acquisitions parameters can include, but are not limited to, LED illumination level and the gain level for an amplifier circuit that is part of the sensor. The applicant has further disclosed this step and how it is performed in U.S. Patent Publication No. US-2021-0353168-A1 which herein is incorporated by reference in its entirety.

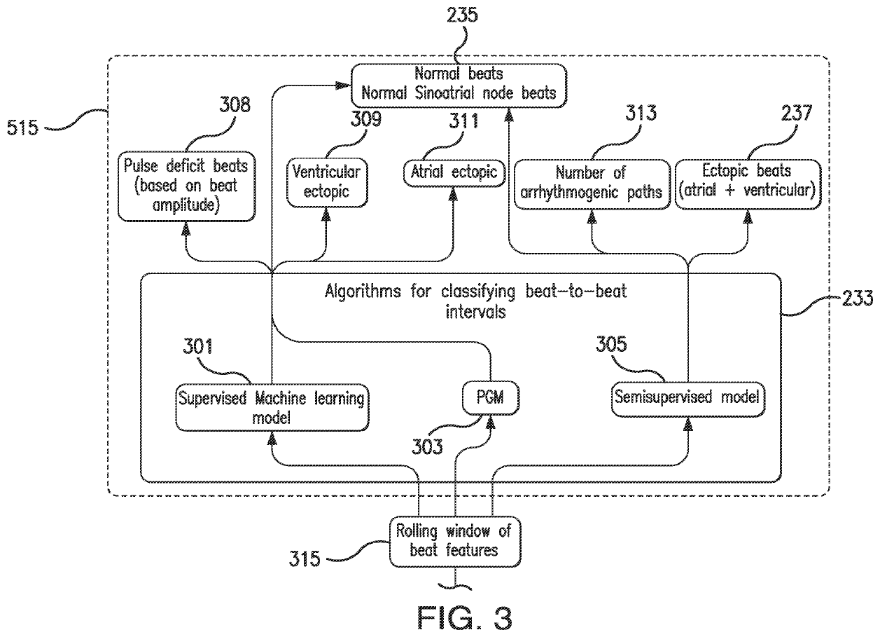

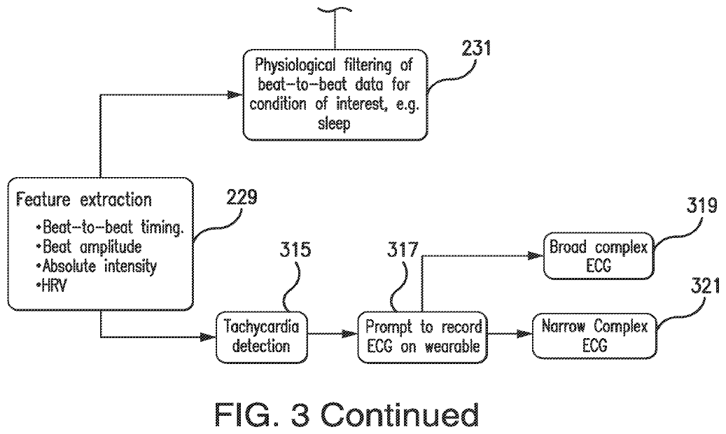

In addition to the sensors 501 for tracking the peripheral pulse, the noninvasive health monitoring device 203 is also outfitted with contextual sensors 503, capable of capturing motion data via actigraphy using a MEMS device (Micro Electro Mechanical System), such as but not limited to triaxial accelerometers and triaxial gyroscopes. The purpose of this data is to monitor the activity of the user, which could be indicative of different physiological states, such as sleep and exercise, but could also be used by the signal processing system(see FIGS. 2 and 3) to remove motion artifacts(see 221 of FIG. 2) that distort the peripheral pulse signal (first generated in 215 of FIG. 2) using applicable techniques known in the art such as using an adaptive filter (see 225 of FIG. 2) which subtract the motion signal from the PPG signal to produce an output signal with minimized noise, as described in Pollreisz, D., TaheriNejad, N., Detection and Removal of Motion Artifacts in PPG Signals, Mobile Netw. Appl. (2019). https://doi.org/10.1007/s11036-019-01323-6 (by minimizing the power of the output signal).

The microcontroller 505 can also perform the process of extracting biological information 229 (see FIG. 2) such as the timing of individual heartbeats from the pulse sensor 501 readings, or it can transmit the pulse sensor readings to another computing device 513 and/or server 515, which can complete this task. In the case where the computation is done via the microcontroller 505 on the device 203, the computed biological information 229 will also be transmitted to other computing devices/servers 513, 515 connected to the internet through a communication module 507 of the wearable device 203 which could comprise any suitable technology including Bluetooth or WIFI. The non-invasive health monitoring device 203 might also possess a communication module 511that is capable of directly communicating with data networks, such as cell towers 517 (see FIG. 5).

### Ectopic Beat Types and Influence on the Peripheral Pulse Waveform

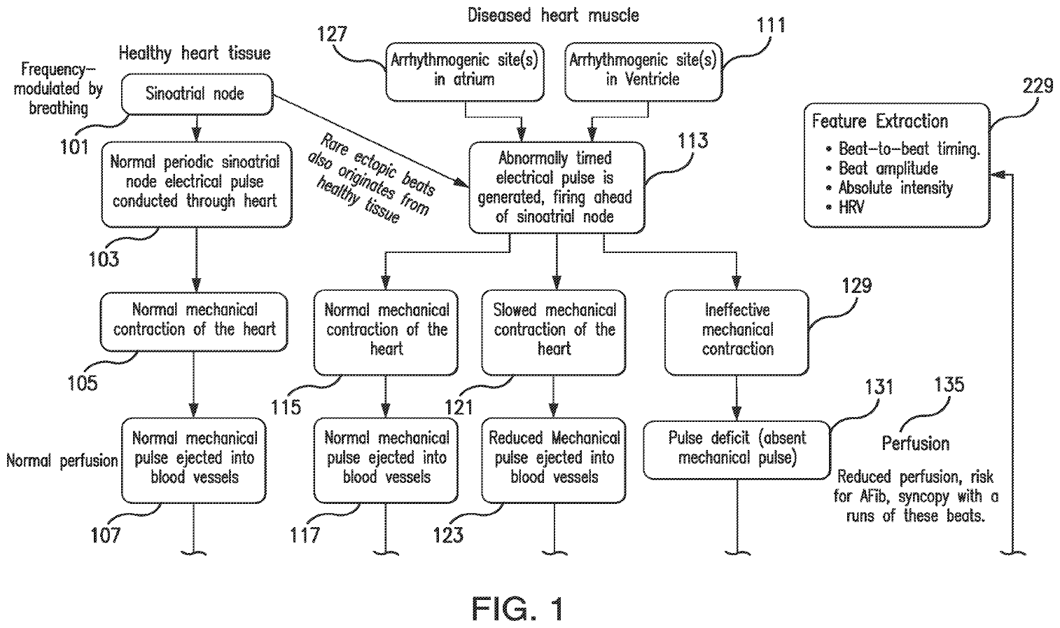

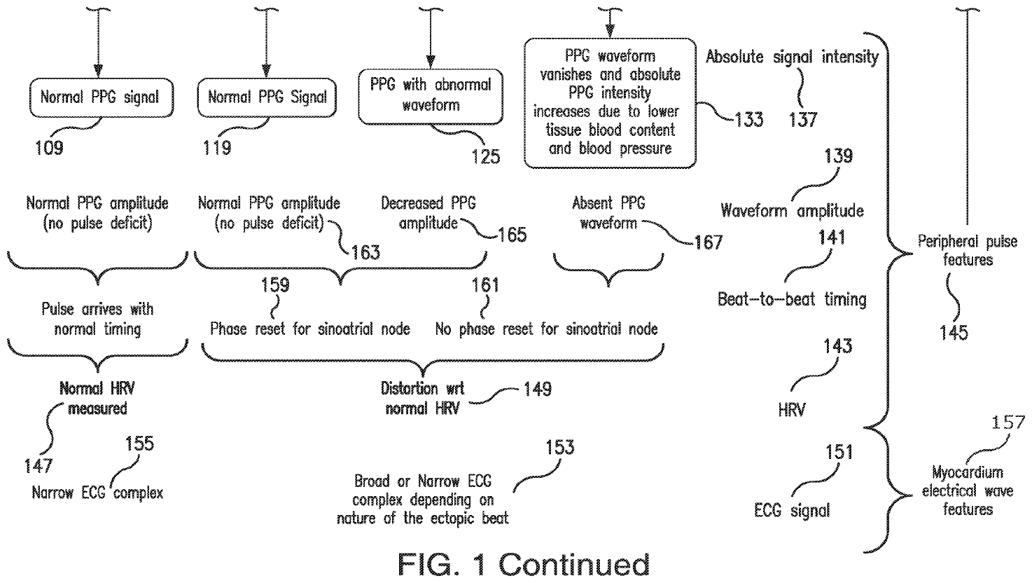

Before covering how the peripheral pulse signal 201 is processed and how it will be used to the benefit of end-users, patients, and clinicians, we describe here how the physiology of normal heartbeats 235 and ectopic heartbeats 237 translate into changes to features of the peripheral pulse signal 229. A healthy heart generates rhythmic beats that follow a predictable pattern, where the time delay between successive heartbeats is similar. These heartbeats originate from the heart's pacemaker cells or sinoatrial node 101, which are conducted throughout the heart at a fast rate (e.g., normal periodic sinoatrial) via a node electrical pulse conducted through the Purkinje fiber network of the heart(see 103), resulting in a narrow ECG QRS complex 155 (see FIG. 1). The heart muscle contracts in response to this electrical wave and generates a normal mechanical contraction 105 which ejects a normal mechanical pulse of blood into the blood vessels 107. This pulse travels from the aorta down to the capillaries at the back of the wrist where a non-invasive health monitoring device 203 can record the resultant waveform of the normal PPG signal 109.

Referring to FIG. 1, an analysis can be performed on a run of such of normal heartbeats 235 (see FIG. 2) using several measures of normal heart rate variability 147, including time domain, frequency domain and entropy-based calculations. For the frequency domain methods, for example, the high frequency band energy (˜0.2 Hz) indicates rhythmic lengthening and shortening of heartbeat intervals in tune with inhalation and exhalation due to a phenomenon known as sinus arrhythmia. This high frequency signal is associated with a series of healthy heartbeats originating from the sinoatrial node 101.

In contrast to normal pulse generation 101, diseased heart tissue in the heart's atrium 127 or ventricle 111 can generate electrical signals that depolarize the heart and cause the generation of an abnormal electrical wave 113 in the heart muscle, known as an ectopic beat, similar to what is seen with normal healthy heartbeats originating in the sinoatrial node 101, as shown in FIG. 1. One difference is that such an ectopic beat precedes the firing of the sinoatrial node 101 which would otherwise have initiated a normal sinoatrial beat. There are also other differences in such an ectopic beat 237 (as identified by digital signal processing and extraction of pulse waveform features found in FIG. 2) compared to a normal sinoatrial beat 101. These differences depend on properties of the ectopic beat, such as whether it originates from the atria (PAC's) or ventricles (PVC's) of the heart. Beats originating in the atria 127 tend to depolarize and reset the pacemaker cells of the heart (i.e., reset to a normal mechanical contraction of the heart (115), reset to a normal mechanical pulse ejected into the blood vessels (117), produce a normal PPG signal (119) with a normal amplitude (163), resetting the phase for the sinoatrail node (159). This causes the phase of subsequent normal heartbeats to change and be in sync with the new beat phase established by the ectopic beat. Such a phase shift can be seen when performing a Fourier analysis of the beat-to-beat time series 141 (FIG. 1), 229 (a component of feature extraction of FIG. 2) and looking for a shift in the phase of the heart rate frequency band. The waveform for such a PAC pulse, when it is not triggered too early in the cardiac cycle, exhibits a comparable amplitude to that of a normal heartbeat, indicated by a normal PPG signal119.

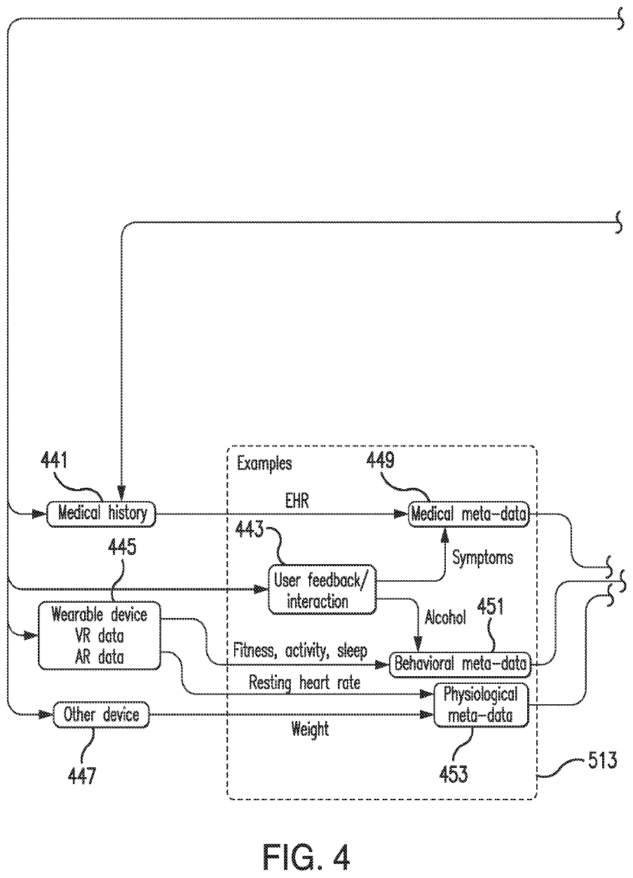

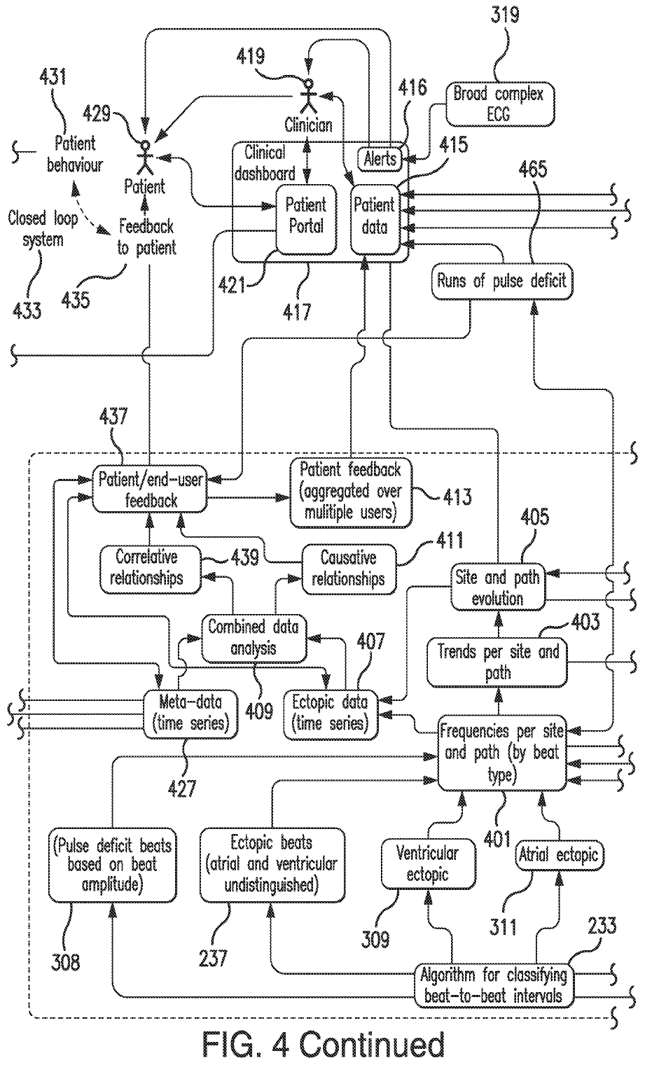

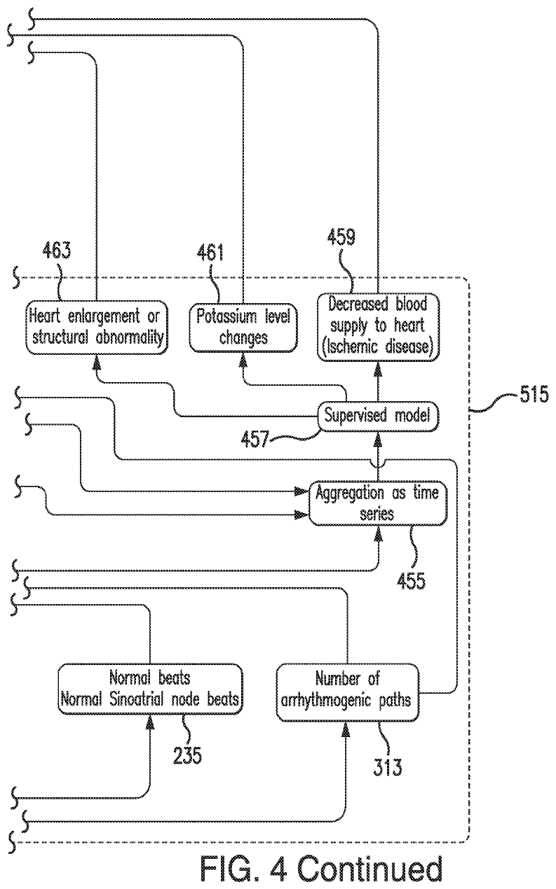

In contrast, beats originating from the ventricles 111, as shown in FIG. 1, often spread too slowly through the heart to reset the pacemaker cells as the electrical wave only hits the pacemaker cells when they are in a refractory phase. This also leads to a ‘compensatory pause’ for normal heartbeats following a PVC, which have an extended delay. This has a direct influence on beat-to-beat timing interval data 141 (FIG. 1), 229 (a component of feature extraction of FIG. 2) collected from the non-invasive peripheral pulse monitoring device 203 and using a Fourier analysis, it is possible to see the preserved phase of normal heartbeats following a PVC. The reason for the slow movement of the PVC wave is that it is not conducted through the high speed Purkinje fiber network connected to the sinoatrial node 101, and therefore the ECG signal/waveform 151 for such a PVC beat is also broadened (153), making it easy to distinguish from PACs that have a narrow ECG complex 155, as well as normal heartbeats originating from the sinoatrial node 101 as both of these have narrow complex ECG waves 155, all of which are myocardium electrical wave features 157. Additionally, due to the abnormal depolarization pattern where the ventricles depolarize before the atria (opposite for normal beats and PAC beats), the heart's normal pattern of first contracting the atria and then ventricles can become disrupted, resulting in an ineffective mechanical contraction 121/129, and result in an inefficient ejection of a blood pulse 123 into the aorta during a PVC. When this effect manifests, a pulse deficit (absent mechanical pulse) 131 can occur as well, and the waveform amplitude 139 of the peripheral pulse PPG can become low (PPG with abnormal waveform125PPG waveform vanishes and absolute PPG intensity increases due to lower tissue blood content and blood pressure 133,decreased PPG amplitude165) or absent (PPG waveform becomes absent 167). In addition, when one or more such beats occur, blood pressure temporarily drops and the absolute PPG signal intensity 137 increases due to a slightly reduced amount of blood in the capillaries monitored by the non-invasive wearable device 203. This phenomenon can also be monitored to determine the presence of dangerous arrhythmia emergencies such as ventricular tachycardia (e.g., 315 of FIG. 3), where many fast beats of this nature occur in series 465 (see FIG. 4;), which can indicate perfusion 135 (FIG. 1, a reduced perfusion, risk for Afib, syncopy with a runs of these beats).

### Digital Signal Processing and Extraction of Pulse Waveform Features

The peripheral pulse sensor readings collected on the non-invasive monitoring device 203 are continuously monitored and the sensor circuit parameters like gain (also LED light intensity) are updated continuously to maximize signal quality as referenced in U.S. Patent Publication No. 2021-0353168-A1 which herein is incorporated by reference in its entirety. The peripheral pulse data is then transformed into a form that will be more continuous and interpretable than a pure sensor reading. Using PPG, for example, the first step would be to calculate the LED and photodiode current level time series 217 and to express the ratio of light received to light emitted 219, as shown in FIG. 2. In this form, the data indicates the absorbance of the skin, which remains constant independent of changes to gain or illumination levels. Similar transforms can be leveraged for other peripheral pulse measurement technologies like SCG 205, BCG 207, ICG 209 or EDA 211.

The non-invasive peripheral pulse measurement device 203 also contains contextual sensors 503 that include actigraphy readings for removing motion artifacts from the signal. Pulse sensors 501, including PPG, SCG, BCG, ICG and EDA (See FIG. 5), are sensitive to motion artifacts, and it is possible via techniques such as adaptive filtering, to remove motion artifacts from the signal 221, as shown in FIG. 2. A common technique is to subtract a linear multiple of the motion signal or time derivative of the motion signal from the peripheral pulse signal 201 or it's derivative respectively in the time domain, while adjusting the multiplication coefficient of this linear subtraction by minimizing the power of the signal remaining after the operation.

Despite all efforts mentioned above, it is possible that a device 203 might not be properly positioned over the measurement site. In other cases, the device 203 might not be attached to the wearer. In such cases it is important to mark data as unreadable and halt processing until proper signal quality is re-established. Such an action may be done by alerting the user through haptic, audio, or electronic messaging feedback 437, via the device 203 itself. To determine the signal quality, the signal-to-noise ratio 223 (with filtering and regions where signal is discontinuous) (see FIG. 2) is calculated for the peripheral pulse sensor data stream 221 (after removal of the motion signal), by calculating the ratio of the power of low-frequency and/or high frequency sample-by-sample noise 224/228 to the energy remaining in the valid signal frequency bands 226 for detecting the peripheral pulse (0.5-5 Hz). Only when sufficient signal quality is available, is the signal processed to extract individual peripheral pulse waveforms for beat segmentation 227.

The process for segmenting the pulse (beat segmentation 227) relies on finding local minima, maxima, and inflection points in the signal to determine the onset of a new peripheral pulse waveform signal. First order derivative zero crossing points indicate the foot and peak of the pulse waveform, while extreme values for the second order derivative indicate the onset of a new pulse waveform. Using the sample numbers for the boundaries of a single pulse waveform derived from this information, it is possible to segment the signal into discrete, continuous and non-overlapping signal segments. Due to the smooth and continuous nature of the peripheral pulse signal, it is also possible to obtain an increased resolution on the exact onset of each pulse waveform that is of higher resolution than the timing between successive peripheral pulse signal samples recorded 215.

Based on the segmenting process (227) discussed above, extracting a group of features 229 from the peripheral pulse features 145 including beat to beat timing 141, waveform amplitude 139, HRV 143, and absolute intensity of the peripheral pulse signal 137 for each peripheral pulse waveform also become available. By calculating HRV 143 on a series of, for example, one minute of beats intervals surrounding the current beat. In this manner it is possible to get an HRV value recorded that can be associated with each beat. Pulse waveform signals can also be stored as features for use in machine learning models for each beat, although some normalization would be required in many cases to convert waveforms of different duration to the same dimensions as model inputs.

Wearable devices 203 can calculate several high-level physiological states such as sleep/wake state, sleep stages, exercise and activity events (see 239 of FIG. 2). Whether derived from the non-invasive peripheral pulse monitoring device 203 or other commercially available wearable devices, this information can be stored as meta-information alongside data on individual heartbeats to provide context on lifestyle factors that might correlate with or be causative with respect to increases in ectopic beat frequencies in an individual. This meta-information is also useful for filtering the time series of heartbeat timings 229, such that for example, only features 229 recorded during sleep, which is probably the least confounded physiological condition (no activity or environmental stimulus), are utilized to analyze (see 231) the frequency and trends in ectopic beats.

### Algorithms for Classifying Beat-to-Beat Intervals

Once available, a time-series of features describing individual heartbeats 231 can be used to distinguish normal and ectopic beats from one another using an algorithm 233 running on a computing device 515, in communication with the wearable device 203 via a network (e.g., internet), as shown in FIGS. 2 and 3. In other embodiments, a wearable device 203 with sufficient computing resources can run the algorithm 233 to distinguish the normal and ectopic beats. As a first stage, the features extracted from individual pulses 231, are aggregated into a rolling window consisting of a fixed number of beat intervals, in the range of a few beats to single minutes of data 315 (see FIG. 3). This window serves as input to any predictive algorithms (e.g., supervised machine learning model 301, a probabilistic graphical model (PGM) 303, and semi-supervised model 305), to produce predictions on whether the newest beat in the rolling window 315 is an ectopic (308, 309, 311, 313 and 237) or normal beat 235.

For example, a supervised machine learning algorithm 301 that has been trained and validated on data of normal peripheral pulse signals aligned with 24-hour ECG data scored by FDA or other appropriately approved algorithms as ectopic beats of atrial or ventricular origin (PACs or PVCs). Two common neural network architectures applicable to this problem are recursive and convolutional neural networks and the relationships discussed above between different types of ectopic beats and their extracted pulse waveform features 229, which provide human interpretable differences between firstly ectopic (308, 309, 311, 313 and 237) and normal beats 235 and secondly between atrial 311 and ventricular 309 ectopic beats and thirdly between normal beats 235 and beats that produce a pulse deficit 308, as shown in FIG. 3. As mentioned above, the sinoatrial node and it's reset with premature atrial beats (PACs) cause a timing difference and phase reset, while retaining mostly normal amplitudes. In contrast, PVCs lead to a compensatory pause and no reset in the sinoatrial node (161), manifest in beat to beat timing differences, and also shows a different pattern with amplitude changes as the slower abnormal electrical wave conduction of a PVC can lead to a strongly attenuated pulse waveform.

A probabilistic graphical model (PGM) 303 can be utilized as well. The PGM 303 represents explicitly the activity of the sinoatrial node as a process with a more periodic timing, with small deviations from the average heart rate (including the known sinus arrhythmia modulation) and the arrhythmogenic tissue as a process with a more uniformly random timing. PGMs deal with conditional dependencies. The state of a prior beat, such as PVC, makes the model rely on a conditional probability distribution where the expected beat timing distribution from the sinoatrial node 101 for the next beat is conditional on the earlier PVC. We can designate this as p(beat-to-beat timelPVC→normal). In p(timing |PVC→normal) the expected or mean beat-to-beat interval for the distribution would be higher than a normal sinoatrial beat representing the known ‘compensatory pause’ for PVCs. For a new datapoint entering the model as it moves through successive beats to classify them, if the beat is indeed extended compared to earlier beats, the probability for the beat-to-beat time when entered into the p(beat-to-beat time PVC— normal) distribution would be higher than for example two normal beats following on one another, designated p(beat timing normal→normal). The PGM 303 includes a set of disjoint beat classes: ‘normal’, ‘PAC’, ‘PVC’. The likelihood for each type can be assessed from their conditional probability distributions. To write out one example, for the case of considering an earlier normal beat, the probability for a given beat-to-beat time for each three of these cases would be the distributions p(beat-to-beat time|normal→PVC), p(beat-to-beat time|normal PAC), p(beat-to-beat time|normal→normal). The most likely of these three would have the highest probability value or likelihood and this would be a function of the beat timing.

When the PGM 303 is evaluating two beats at a time, all 9 transitions (3×3 transitions over normal, PAC and PVC) would be considered and the pair with the maximum likelihood for successive beats, e.g. p(beat-to-beat time ‘-1’ |normal→PVC)×p(beat-to-beat time ‘-2’ |PVC→normal), would be used to determine the classification output of the model, where ‘-1’ designates the most recent beat-to-beat timing and ‘-2’ the second most recent. Similarly, we can also assess such probabilities for other beat features, such as the amplitude of the beat. For example, the attenuated beat amplitude of a PVC would be represented by a lower mean value for the amplitude of the waveform in the distribution p(beat amplitude|normal→PVC). When considering the overall likelihood for classification using the different features, the probabilities for each feature type are multiplied. For example, considering only the most recent beat-to-beat interval, when using two features, beat-to-beat timing and beat amplitude, we would state the likelihood function as the product of these two e.g.: p(beat-to-beat time normal→PVC)×p(beat amplitude|normal→PVC). This process is trained by building the conditional probability distributions on data from simultaneously recorded PPG and ECG data as highlighted

in and can be extended to consider longer transition sequences, such as those occurring over three or more beat sequences (e.g. p(beat-to-beat time|PVC→p normal —p normal)). This strategy can also be trained using data from patients with runs of ectopic beats that occur in a distinct pattern due to downstream effects of an initial ectopic beat trigger 313.

Beyond the supervised model 301 and PGM 303, it is also possible to add a Semisupervised Model 305, which contains a step for automatically finding clusters of dense, similar beat patterns. In an aspect, a relatively short rolling window of beat features can be used, ranging from three to six beats, to automatically discover sequences of ectopic beats that sometimes occur in regular patterns 313 in individual patients, as shown in FIG. 3. The space to be clustered by the algorithm, is the multidimensional space of dimensions 2-6 beats, termed the Phase Space. The Poincare plot is an example of this Phase Space of dimensions 2. Common examples of this are arrhythmia conditions of bigeminy and trigeminy where a beat sequence is regularly seen that consists of two or three abnormal beats. The approach is to firstly identify clusters in the multidimensional beat sequence space mentioned, to subsequently mark all clusters close to the diagonal (where all three to six dimensions have a similar value) as normal beats. The following step is then to determine the number of clusters that were formed by abnormal clustered activities on the arrhythmia phase space and listing each as contributing to the total number of arrhythmogenic paths (313) (see FIG. 4). By tracking the evolution in the number of these clusters 313 and that represent arrhythmogenic paths as well as the beat frequency 401 and beat trends in these clusters 403, a more complete picture of the evolution 405 is obtained of how the heart might be changing to become more or less prone to generating abnormal beats due to underlying disease processes 407, as shown in FIG. 4.

### Monitoring and Management System

Up to this point the process for finding and classifying ectopic beats and producing information on their evolution across frequency, different types (PACs, PVCs and pulse deficit beats) 308, 237, 309, 311, 401 and across different arrhythmogenic paths 313, 401 using the non-invasive peripheral pulse monitoring device 203 has been covered. Using this information several routes for leveraging the early disease marker of ectopic beats for early monitoring and management of cardiac health exists: (1) Screening for specific underlying causes of ectopic beat frequencies and trends (2) Screening for and managing runs of ectopic beats 3.) Determining behavioral and environmental factors that influence ectopic beat frequencies 4.) Opportunistically prompting the collection of ECG information.

### Screening for Specific Underlying Causes of Ectopic Beat Frequencies and Trends

We have described a supervised machine learning model 301 as an algorithm for classifying beat-to-beat intervals 233 above. We have also mentioned the use of reference ECG based classifications of ectopic beat types and peripheral pulse data to train this model. Public databases of ectopic beat types recorded under conditions such as heart enlargement or structural abnormalities 463, potassium level changes 461, Ischemic disease (decreased blood supply to heart)459 (See FIG. 4) and coronary artery disease are available on public resources like Physionet (https://www.physionet.org/data/). Using the same strategy for training the model as discussed above, a second supervised machine learning model 457 can be used to classify the feature information extracted from the non-invasive peripheral pulse on ectopic beats from the aggregation as time series information 455 (e.g., frequencies per site and path, by beat type, 401, trends per site and path 403, and site and path evolution 405) in terms of a possible cause 462, 461, 459. This information is then uploaded to the patient data section 415 of a digital platform 417 for managing and interacting with patients 429 and registered to a cardiologist 419. Based on the interpretation of the cardiologist 419, follow up actions, either clinical or lifestyle based, can be shared with the patient through the patient portal 421.

### Screening for and Managing Runs of Ectopic Beats

Similarly, information for the site and path evolution 405 of ectopic beats, is also uploaded to the patient data section of the digital platform 417. Based on the interpretation of the cardiologist 419, follow up actions either clinical or lifestyle based, can be shared with the patient 429 through the patient portal 421. The system can utilize behavioral meta-data 451 (e.g., sleep meta-data), to provide context on when and under which conditions these runs of ectopic beats occurred. In addition, physiological meta-data 453, collected from other devices 445/447, can be utilized as well. It has become known for example that athletes suffer from a higher incidence of atrial fibrillation (AFib) and that this manifests under conditions of high vagal and low sympathetic activity, such as deep sleep. By sharing this information with the cardiologist, arrhythmias and ectopic runs that might never be recorded during a daily clinic visit can be put under the attention of a cardiologist for treatment.

### Determining Behavioral and Environmental Factors that Influence Ectopic Beat Frequencies

An important second source of information, meta-data 427 on the behavior 431 and environment of the patient, is collected and aggregated on a computing device c 513/515 with communication means. In an aspect, this information is aggregated through a user interface, which can be presented on a second computing device 513 connected to the internet such as a mobile phone, to collect user feedback 443, a server 515 that connects to APIs for device 203, or another wearable device 445 similarly capable of measuring user behavior 431 (e.g., collecting VR and AR data), as well as other devices 447 in the user's environment capable of measuring physiological information such as the patient's weight, as well as medical meta-data 449 that is gathered from sources such as an electronic health record (EHR) (i.e., medical history of the patient) 441imported into the system 515 or disclosed by the patient via the app on a local computing device 513 (e.g., a smart phone). Additionally, patients can manually log their information or smart pill containers can automatically fill out information on day-to-day dosage of different pharmaceuticals. The secondary wearable device 445, for recording physiological information, could also include information from devices such as virtual reality or augmented reality hardware, capable of performing eye and body tracking data and measuring activity and behavioral information for virtual environments (such as lighting levels as a simple example).

The aggregated meta-information 427 and the ectopic beat information/data 407, which can be in time series, combined and analyzed 409, that includes trends 403, site and path evolution 405. This step provides calculations that determine the correlation between different ectopic beat trends and trends in meta information such as medication type and usage. Correlated trends are then presented to the patient, to inform her on the effects of various behavioral and environmental factors on ectopic beat trends. This information is also uploaded to the patient data section 415 of a patient management platform 417 to provide the patient's cardiologist with information on significant environmental factors that might be influencing cardiac health, which could prompt further investigation and or lifestyle and medication changes. Causative trends 411 are generated by studying lagged correlations where prior behavior delayed by, for example, one week is correlated against more recent ectopic beat information 407 to discover correlations that are more likely to be causative. This information is used in a similar manner as described for correlative trends/relationships 439, packaged at a low level of detail/feedback 437 for sharing with the patient and at a higher level of detail/patient feedback 413, which can be aggregated over multiple users, for sharing with the cardiologist/clinician 419. A closed loop feedback system 433 is created by this system where the patient 429 continuously receives feedback 435 on her cardiac health at a very fine resolution, capable of warning the user of very early changes, which are the easiest to correct through lifestyle and medical intervention, while also informing her as to the measured trends in her behavior and environment that coincide with changes to her ectopic beat trends and frequencies 407. As simple examples, the system could warn of unintended pro-arrhythmogenic effects of new medications or new dietary supplements, as well as the influence of parameters such as decreasing weight on heart health. Similarly, the system can detect healthy behaviors such as exercise, whereas this will show increases in cardiac health as measured through ectopic beat data 407 in most cases, for very specific cases; hypertrophic cardiomyopathy exercise, for example, might lead to increases in ectopic beat frequencies, which a cardiologist should be aware of.

### Opportunistically Prompting the Collection of ECG Information

Finally, runs of ectopic beats discovered by the non-invasive peripheral pulse monitoring device 203, can be used to prompt users to collect and record ECG information 317 (e.g., after tachycardia detection 315), by, for example, touching the electrode exposed on a wearable device 203, 445 with a finger of the opposing hand. In cases where such a run of ectopic beat data has broad complex ECG patterns 153/319, an alert 416 can be generated to warn the cardiologist 419 and patient 429 of the occurrence of potentially deadly ventricular tachycardia. In addition, runs of abnormal beats 465 can further inform on the risk or as historic information, the cause for stroke and syncope and this information is also uploaded to the patient management system 417 for follow up actions.

What is claimed is:

1. A method to perform non-invasive health monitoring, comprising:
a. acquiring data on a peripheral pulse of a subject through the use of non-invasive wearable;
b. analyzing the data collected by;
i. extracting and classifying heart beats using a predictive algorithm; and
ii. predicting other contextual information using data from the wearable device; and
iii. analyzing trends in ectopic heart beat frequency; and
c. sharing the ectopic beat frequency and trends from the analysis with patients and care providers.

2. The method of claim 1, wherein the wearable device comprises a sensor capable of monitoring the peripheral pulse and a contextual sensor.

3. The method of claim 2, wherein the peripheral pulse sensor utilizes photoplethysmography (PPG), seismocardiography (SCG), ballistocardiography (BCG), impedance cardiography (ICG), or electrodermal activity (EDA).

4. The method of claim 2, wherein the contextual sensor comprises a triaxial accelerometer, a gyroscopic sensors, or electromyography.

5. The method of claim 2, wherein the wearable device is configured to:
a. a. filter the signal to remove low quality recordings using readings from contextual sensors to determine whether motion is present;
b. b. use readings from peripheral pulse sensors to determine whether the signal to noise ratio is acceptable; and
c. use a threshold for detecting discontinuities in the peripheral pulse signal that could be due to adjustments in the signal amplification parameters or due to noise sources.

6. The method of claim 1, wherein the predictive algorithm is configured to identify:
a. extracted heartbeats as normal beats, initiated by the sinoatrial node or pacemaker of the heart;
b. ectopic beats, not initiated by the sinoatrial node or pacemaker of the heart; or
c. undetermined beats.

7. The method of claim 6, wherein the predictive algorithm is further configured to takes as input the number of ectopic beats, trends in the number of ectopic beats, and features describing individual ectopic beats.

8. The method of claim 7, wherein the predictive algorithm can produce a prediction for the cause of ectopic beats in cases where these are elevated, or changing or trending over time, wherein the potential causes include heart enlargement or other myocardial abnormalities, changes in potassium level, or decreased blood supply to the heart.

9. The method of claim 1, wherein the wearable device is configured to convert the peripheral pulse signal to a unit that would retain continuity in the peripheral pulse signal across signal acquisition parameter adjustments.

10. The method of claim 1, further comprising segmenting individual peripheral pulses from the peripheral pulse signal readings, the segmenting comprising:
a. taking a derivative of the peripheral pulse and using zero crossings to locate peaks and/or troughs of the signal; and
b. taking a second order derivative of the peripheral pulse to locate inflection points.

11. The method of claim 10, wherein the time resolution of pulse peaks and/or troughs is increased beyond the time resolution of the sampling rate by performing interpolation of the signal and finding the peak or trough in the interpolated signal utilizing a polynomial interpolation and a spline interpolation.

12. A method for non-invasive health monitoring, comprising:
a. acquiring data on
i. a peripheral pulse of a subject, using a non-invasive wearable device comprising:
1. at least one microcontroller;
2. sensors capable of monitoring;
a. the peripheral pulse, such as, but not limited to:
i. photoplethysmography (PPG);
ii. seismocardiography (SCG);
iii. ballistocardiography (BCG);
iv. impedance cardiography (ICG); or
v. electrodermal activity (EDA);
b. contextual sensors, such as, but not limited to:
i. triaxial accelerometer;
ii. gyroscopic sensors; or
iii. electromyography,
3. a communication module for sending recorded data to a computing device connected to the internet,
ii. optional demographic information of a subject via an electronic device, including, but not limited to:
4. Height;
5. weight;
6. BMI;
7. Sex; and
8. age,
iii. optional medical history of a subject by means of Electronic Health Record of similar,
b. transmitting the acquired data to one or a series of computing device, such as, but not limited to, mobile phones, servers, tablet computers, the computing device connecting to either:
iv. the internet; or
v. local communication network, such as bluetooth or wifi, that can reach the internet through other devices,
c. analyzing the acquired data
i. by extracting and classifying heart beats using a predictive algorithm as either;
1. normal beats, initiated by the sinoatrial node or pacemaker of the heart;
2. ectopic beats, not initiated by the sinoatrial node or pacemaker of the heart; or
3. undetermined beats,
ii. by predicting other contextual information using data from the wearable and the optional demographic data, including, but not limited to:
1. sleep state and sleep stages;
2. activity patterns; and
3. fitness level,
iii. by storing and analyzing trends in ectopic heart beat frequency,
d. transmitting information on ectopic beat frequency and trends to:
i. patients, to inform them on their progress toward improved or worsening heart health at the earliest or later stages of disease; and
ii. care providers to track:
1. disease progress;
2. Treatment efficiency; and
3. Lifestyle intervention efficiency.

13. The method of claim 12, wherein the ectopic beat frequencies and trends are transmitted to:
a. patients, to warn them of runs of ectopic beats associated with a reduced pulse or pulse deficit to be used for, but not limited to,
i. timely visit to the ER; and
ii. reaching out to their physician,
b. care providers to
i. alert with regard to periods of pulse deficit that predispose the subject to
1. stroke; or
2. syncope, and
ii. to show historical periods of pulse deficit to determine whether a stroke might have been caused by runs of ectopic beats/arrhythmia.

14. The method of claim 13, wherein runs of ectopic beats detected in athletes during sleep are used to enable early detection of atrial fibrillation that only manifests under conditions of high vagal tone, such as deep sleep.

15. The method of claim 12, wherein the total number of ectopic beats or trends in the historic frequency of ectopic beats in a subject are displayed to at least one of:
a. a clinician, to
i. inform patient screening by highlighting patients that have a high or increasing ectopic burden; and
ii. monitor the condition of the subject including:
1. monitoring the efficiency of treatment;
2. monitoring the number of arrhythmogenic sites; or
3. monitoring the compliance and efficiency of lifestyle interventions such as exercise through the data recorded; or
b. a patient to inform them of:
i. ectopic burden with threshold alerts to seek medical care;
ii. trends in ectopic burden with threshold alerts to seek medical care; and
iii. the relationship between the metadata, ectopic burden, and ectopic trends to improve wellness, wherein said metadata includes:
1. sleep parameters;
2. exercise and activity parameters such as:
a. number and amount of exercise per week,
b. sedentary behavior such as longest consecutive sitting, time per day, and
c. steps taken per day; and
3. body weight.

16. The method of claim 12, wherein the wearable device or computing device is connected to the internet and is configured to:
a. determines whether tachycardia is present from the peripheral pulse rate using digital signal processing techniques;
b. alerts the user to record an ECG using the wearable device by touching a finger to an electrode on the device when tachycardia is detected;
c. determines from the ECG data whether the QRS complex is a broad or narrow complex; and
d. alerts, following to the presence of ventricular tachycardia in the case of broad complex QRS, emergency services, the patient, or the patient's care provider.

17. The method of claim 12, wherein the method comprises an algorithm configured to:
a. takes as input:
i. the number of ectopic beats;
ii. trends in the number of ectopic beats; and
iii. features describing individual ectopic beats, such as, but not limited to the pulse attenuation or pulse deficit,
b. produces a prediction for the cause of ectopic beats:
i. in cases where these are:
1. elevated, or
2. changing or trending over time,
ii. where the potential causes include, but are not limited to:
1. heart enlargement or other myocardial abnormalities;
2. changes in potassium level; and
3. decreased blood supply to the heart (ischemic disease).

18. The method of claim 12 further comprising:
a. signal acquisition parameters, including, but not limited to amplifier gain and LED current which are adjusted dynamically during measurement through a closed loop controller to continuously optimize the signal to noise ratio for the peripheral pulse signal;
b. converting the peripheral pulse signal to a unit that would retain continuity in the peripheral pulse signal across signal acquisition parameter adjustments such as, but not limited to:
i. PPG, where a unit would be the ratio between light emitted by the photodiode and light received;
ii. BCG, where a unit would be acceleration measured in G;
iii. impedance, where a unit would be the complex resistance for a given frequency; or
iv. galvanic skin response, where a unit would be the resistance.

19. The method of claim 12, wherein the signal is filtered to remove low quality recordings, the method comprising:
a. using readings from contextual sensors to determine whether motion is present, which would distort signals;
b. using readings from peripheral pulse sensors in to determine whether the signal to noise ratio is acceptable, wherein the signal to noise ratio can be determined through any of the following means of comparing the:
i. ratio of high frequency sample by sample noise to signal in frequency bands corresponding to measured heart rate, or
ii. ratio of signal energy in the frequency bands corresponding to heart rate to other frequency bands;
c. using a threshold for detecting discontinuities in the peripheral pulse signal that could be due to adjustments in the signal amplification parameters or due to noise sources.

20. The method of claim 19, further configured to remove noise from the signal by:
a. filtering the signal to remove high frequency noise by application of a low pass filter that removes sample by sample noise; and
b. filtering the signal to remove low frequency noise by application of a low pass filter that removes noise that includes, but is not limited to physiological processes, such as breathing,
wherein band-pass filtering is utilized to remove both the high frequency noise and the low frequency noise.

21. The method of claim 12, further comprising segmenting individual peripheral pulses from the peripheral pulse signal readings, the segmenting comprising:
a. taking a derivative of the peripheral pulse and using zero crossings to locate peaks and/or troughs of the signal; and
b. taking a second order derivative of the peripheral pulse to locate inflection points.

22. The method of claim 21, wherein the time resolution of pulse peaks and/or troughs is increased beyond the time resolution of the sampling rate by performing interpolation of the signal and finding the peak or trough in the interpolated signal utilizing a polynomial interpolation and a spline interpolation.

23. The method of claim 22, wherein individual pulses are reduced to a set of features relevant for discriminating ectopic beats from beats originating from the sinoatrial node, with the set of features further comprising beat-to-beat timing, beat amplitude, absolute signal intensity for the trough of the signal, heart rate variability based on surrounding beats for approximately one minute, and pulse waveform features.

24. The method of claim 12, wherein the algorithm for classifying individual beats as ectopic or normal is based on any of the following principles:
a. a supervised machine learning model trained against ectopic beats identified in datasets scored by expert human or algorithm, where said datasets include simultaneous ECG and PPG recordings, wherein said supervised algorithm is trained on population level data or data collected from an individual to provide personalized training of the model;
b. a semi-supervised machine learning model, which produces clusters in the space of the Poincare plot (beat compared to previous beat) and which marks the cluster(s) closest to the diagonal as normal sinoatrial beats, while marking other clusters as ectopic beats; or
c. a Probabilistic Graphical Model (PMG) that models the distribution of beat timing for normal sinoatrial beats as well as for ectopic beats, a variation of the PMG comprising of either:
i. the PGM where the distribution for normal and ectopic beats depends on one or more earlier beats;
ii. the PGM is a Hidden Markov Model (HMNI) with at least one hidden state representing normal and another hidden state representing ectopic beats; or
iii. the PGM is a Bayesian network, where the distribution for normal and ectopic beats depends on at least the previous beat,
wherein maximum likelihood is used to predict the nature (normal or ectopic) of the next beat to beat timing.

25. The method of claim 24, wherein in addition to its status as normal or ectopic, the following information is predicted from the features available for each beat:
a. whether the beat is likely to have originated from either the atrium or ventricle of the heart; and
b. the ectopic beat path, from which the beat originates, with new clusters potentially representing new arrhythmogenic modes in the heart muscle.

26. The method of claim 12, wherein the sensors for following the beat timing of the peripheral pulse are replaced with ECG to follow beat timing via the electric signals of the heart.
